(19) United States
(12) Patent Application Publication
(10) Pub. No.: US 2009/0178173 A1
(43) Pub. Date: Jul. 16, 2009

(54) SPORTS HAND WARMER

(76) Inventor: Andrew Schultz, Parker, CO (US)

Correspondence Address: SHERIDAN ROSS PC, 1560 BROADWAY, SUITE 1200, DENVER, CO, 80202, US

(21) Appl. No.: 12/353,967

(22) Filed: Jan. 14, 2009

Related U.S. Application Data
(60) Provisional application No. 61/020,971, filed on Jan. 14, 2008.

(51) Int. Cl. A61F 9/00; A41D 27/00

(52) U.S. Cl. 2/16; 2/243.1

(57) ABSTRACT

The invention provides a portable hand-warming apparatus for athletes that is easily attached and detached from the athlete's clothing but remains safely unobtrusive with the movement of the player during an athletic event. The apparatus is made of multiple layers of materials to reduce moisture, insulate, and resist wind and water. In one specific embodiment, the apparatus may be used to warm the throwing hand of a baseball or softball player while inactive during play, such as between pitches or while playing in the field.

### CROSS REFERENCE TO RELATED APPLICATION

This application claims the benefit of priority under 35 U.S.C. §119(e) to U.S. Provisional Patent Application No. 61/020,971 filed Jan. 14, 2008, which is incorporated herein by reference.

### TECHNICAL FIELD

The invention relates to hand warmer for athletes participating in cold weather sporting events.

### BACKGROUND OF INVENTION

A common problem for fall and winter athletes is the inability to protect one or both hands in cold weather when their uniforms do not possess any means to keep their hands warm. This can dramatically impact the performance of these athletes in cold weather. It is not typically practical for these athletes to wear a large hand warming apparatus, such as a football hand warmer around the waist. This may be due to performance considerations. For example, a baseball or softball infielder may be prohibited from attempting to catch a ground ball by having to move the hands around a waist hand warmer to make the play. It is also not practical for most athletes to wear a traditional glove or even a batting glove, as this impacts the player's grip on sports gear, including balls, bats, clubs and sticks, which can negatively affect performance, as these athletes need to grip the gear to properly execute the athletic event. For example, a baseball/softball player needs to be able to grip the baseball and feel the laces to make strong and accurate throws, a task that is greatly impaired when executed in cold weather using a cold throwing hand or a throwing hand wrapped in a glove.

Each of the foregoing disadvantages of the prior art apparatuss are overcome by an apparatus of this invention, which has other advantages discussed more fully below.

### SUMMARY OF INVENTION

The present invention allows an athlete to warm a hand while inactive or idle during a sporting event to prevent diminished performance during periods of activity that require the active use of the hand. For example, a baseball/softball player would be able to place the throwing hand in the warmer between pitches while playing defense in the field. This warmer is specifically designed for sports uniforms, enabling attachment by three different methods according to the position of the player.

An advantageous aspect of the invention is that it allows an athlete to determine the most comfortable position to place the hand warmer. For example, as baseball and softball pants do not have loose or hanging articles, it is important the player positions the hand warmer in a functional location enabling quick and flawless entry and exit from the warmer. Apparatuss of the present invention allows for that efficient entry and exit.

Another advantage is the configuration that allows athletes to keep a warmer concealed and secure during play. This is an important advantage to prevent loss of the warmer and to prevent injuries to the athlete should the warmer interfere with the athlete's movement during the sporting event, and the prevention of injuries to other athletes should the warmer become dislodged and loose on a playing field. For example, a baseball/softball player would not need to be concerned with losing the warmer when worn in the pocket or jersey while attempting a slide when running bases.

Additionally, because the warmer is portable and easily removed, it enables an athlete to utilize the same warmer for different uniforms, such as home and road uniforms (jerseys and pants), as well as potentially different uniforms for later seasons.

### BRIEF DESCRIPTION OF DRAWINGS

FIG. 1, shows an embodiment of the present invention deployed around a strap.

FIG. 2, shows a front view of an embodiment of the hand warmer of the present invention.

FIG. 3 shows a side view of the embodiment of FIG. 2.

FIG. 4 shows a top view of an embodiment of the hand warmer of the present invention.

FIG. 5 shows a view of the back of the embodiment of FIG. 2.

FIG. 6 shows a view of the bottom of the embodiment of FIG. 2.

FIG. 7 shows a sectional view of the hand warmer of FIG. 2 at line 7, deployed within an article of clothing.

FIG. 8 shows an embodiment of the hand warmer of the present invention deployed internal to the waist band of an athletic uniform.

FIG. 9 shows an embodiment of the hand warmer of the present invention deployed internal to a pants pocket of an athletic uniform.

FIG. 10 shows an embodiment of the hand warmer of the present invention deployed internal to a jersey of an athletic uniform.

### DESCRIPTION OF EMBODIMENTS

The present invention is drawn to an apparatus that protects or warms the hand(s) of an athlete while remaining securely and unobtrusively affixed to the athlete's clothing during the movement of the athlete engaged in a sporting event. The apparatus is compact and portable and affixed within the athlete's clothing to make the apparatus suitable for use during athletic events without endangering the athlete wearing the apparatus or other athletes that may be around or in contact with the athlete wearing the apparatus. In a preferred embodiment, the apparatus is configured for the purposes of warming the useful hand in athletic competition, such as the baseball and softball throwing hand.

In one embodiment, the apparatus is a portable apparatus composed of a first side and a second side opposite from the first side such that the first and second side together form an enclosure adapted to receive a human hand. One end of the enclosure is a closed end and the opposite end open sufficient to allow entry of a human hand into the enclosure. The open end includes an attachment flap, which is a flap of material connected to one side of the apparatus proximate to the opening in the open end. In a preferred embodiment, the first and second side of the apparatus is composed of three layers: 1) a breathable moisture reducing layer, 2) an insulating layer, and 3) a wind and water resistant layer.

The attachment flap contains an attachment mechanism that is operable to securely engage an article of clothing. The attachment mechanism may be a magnet embedded within or attached to the flap that is attracted to a magnet in one of the layers of apparatus. The attachment mechanism may also be one or more buttonholes that engage an existing button on an athlete's clothing. Conversely, the attachment mechanism may also be one or more buttons that engage an existing buttonhole on an athlete's clothing. The attachment mechanism may also be hook and loop fabric connectors wherein one of the hook and loop connectors are disposed on the attachment flap and the opposite connector is attached to the athlete's clothing. The attachment mechanism may also be at least one snap designed to reversibly attach to a corresponding snap on an article of athletic clothing. The attachment mechanism may also be at least one clip designed to reversibly attach to an article of clothing.

In one specific embodiment, the apparatus is designed to have two flaps near the open end where the hand is inserted. One of the flaps has one or more of the attachment mechanisms described above. The other flap is without an attachment mechanism and may aid in the insertion of a hand into the device.

To be utilized without affecting performance, the apparatus has three primarily preferred methods of use. First, the apparatus may be placed in the back pocket of an athlete's pants or shorts. In this instance, the apparatus is attached to the player's pants by the attachment means. Alternatively, the apparatus may be inserted under one side of an athlete's shirt or jersey and attached through the attachment mechanism to the front of the shirt or jersey. Lastly, the apparatus may be placed around a belt or strap worn by the athlete and the attachment mechanism holds the apparatus securely in place around the belt or strap. In this last instance, the strap may be an athlete's waist band such that the apparatus is tucked within the athlete's pants or shorts and the opening is located proximate the athlete's waist band such that the attachment flap and attachment mechanism resides outside of the athlete's pants and holds the apparatus securely around the athlete's waist band.

In another specific embodiment, the apparatus also has loops on one of the first or second sides into which a strap can be inserted, the strap being useful to attach the apparatus to an arm, leg or torso of an athlete. In this embodiment, the apparatus may be attached to an arm, leg or torso of the athlete by use of a band placed through the fastener and loops and around the athlete.

In one preferred embodiment of the invention a hand warmer is composed of a breathable moisture reducing layer; an insulating layer; and, a wind and water resisting layer such that each of these three layers forms two opposing sides of a hand enclosure having one end open sufficient to allow unobstructed entry and removal of a human hand. This embodiment includes a fabric attachment on one of the two opposing sides of the opening and contains means to attach the hand enclosure to an article of clothing.

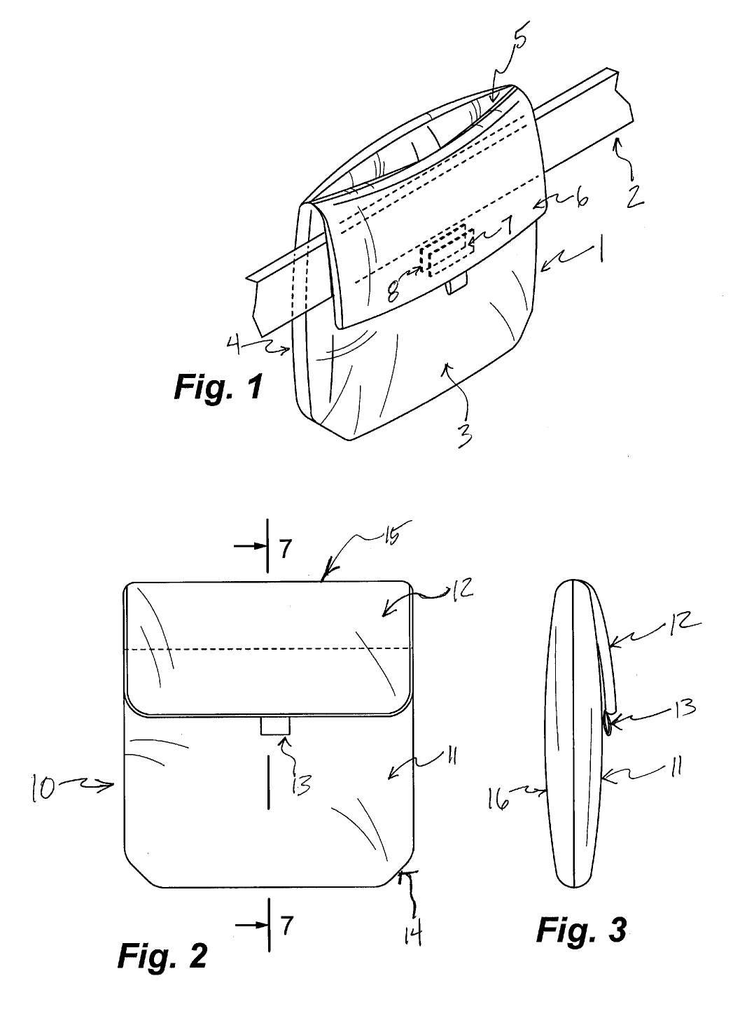

Referring to FIG. 1, a particularly preferred hand warmer (1) of the present invention is shown deployed around a belt (2). The hand warmer (1) includes a first side (3) and a second side (4) opposite the first side (3) and an opening (5) at one end into which an athlete may insert a hand. An attachment flap (6) is attached to the first side (3) proximate the opening (5). Embedded within the attachment flap (6) is a first magnet (7) that is attracted to a second magnet (8) embedded within the first side (3) such that the first magnet (7) and second magnet (8) hold the attachment flap (6) and the hand warmer (1) securely affixed around the belt (2).

Referring to FIGS. 2 and 3, one side (11) of a particularly preferred hand warmer (10) of the present invention is shown with an attachment flap (12) attached near an open end (15) of the hand warmer (10). In this embodiment, the attachment flap (12) contains a fabric loop (13) that can be used to pull the attachment flap (12) away from the athlete's clothing to separate the attachment mechanism and remove the hand warmer (10) from the athlete's clothing. One or more corners (14) of the hand warmer (10) may be rounded as shown in FIG. 2 to allow for easier insertion to the athlete's clothing.

Referring to FIG. 3, a side view of the hand warmer of FIG. 2 shows the second side (16) of the hand warmer opposite the first side (11).

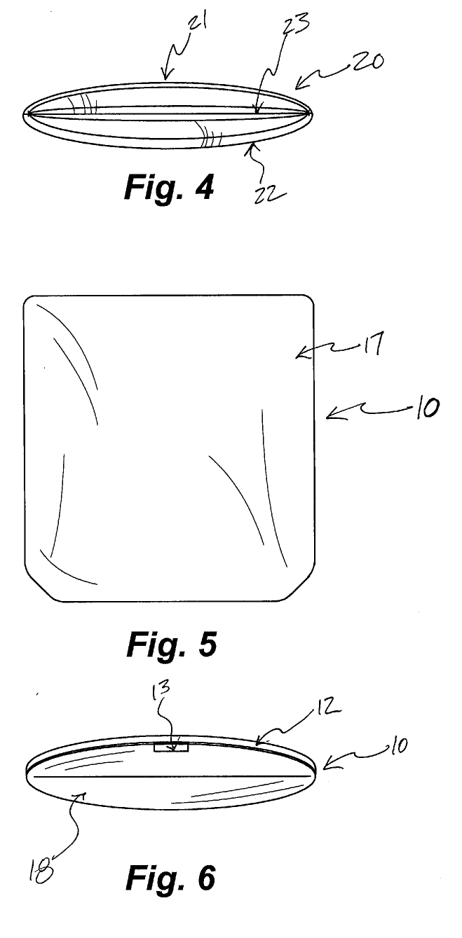

Referring to FIG. 4, a top view of a hand warmer (20) of the of the present invention is shown. A first side (22) and a second side (21), opposite the first side (22) form an enclosure with an opening (23) of sufficient size to receive an human hand.

FIG. 5 shows the back view of the hand warmer (10) of FIG. 2. The second side (17) of the hand warmer (10) is opposite the first side (11, FIG. 2).

FIG. 6 shows a bottom view of the hand warmer (10) of FIG. 2. The bottom of the hand warmer (18) closes the end of the hand warmer (10) opposite the open end. The fabric loop (13) is visible on the bottom of the attachment flap (12).

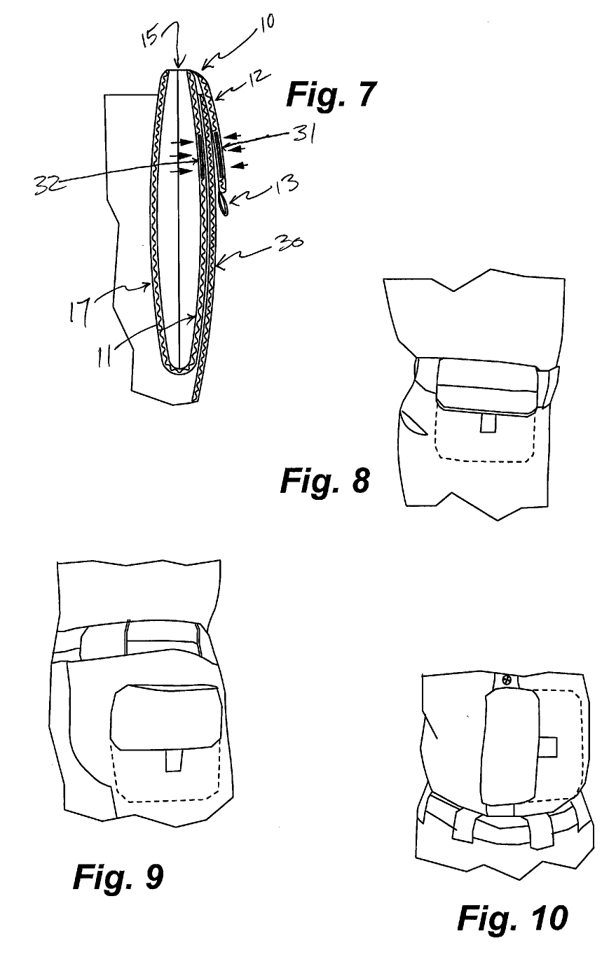

FIG. 7 shows a sectional view of the hand warmer (10) of FIG. 2 about line 7, deployed around an article of clothing (30). The first side (11) of the hand warmer (10) is opposite the second side (17) and is attached to the attachment flap (12) near the open end (15). A magnet (31) is embedded in the attachment flap (12). The magnet (31) is attracted to a magnet (32) embedded in the first side (11) of the hand warmer (10) such that the magnets (31, 32) hold the attachment flap (12) and the first side (11) of the hand warmer (10) securely fastened around the article of clothing (30). The fabric loop (13) may be used to pull the attachment flap (12) away from the first side (11) of the hand warmer (10) to disengage the magnets (31, 32) and remove the hand warmer from the article of clothing (30).

FIG. 8 shows a hand warmer of the present invention deployed within the pants of an athlete around the waist band of the athlete's pants. Similarly, FIG. 9 shows a hand warmer of the present invention affixed within the back pants pocket of an athlete. FIG. 10 shows a hand warmer of the present invention deployed within the jersey of an athlete within an opening in the athlete's jersey.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A hand warmer, comprising:
a first side;
a second side opposite from the first side, wherein the first and second side together form an enclosure adapted to receive a human hand;
a closed end;
an open end opposite from the closed end, wherein the open end includes an opening sufficient to allow entry of a human hand into the enclosure;
an attachment flap connected to the first side proximate to the opening; and,
an attachment mechanism associated with the attachment flap, wherein the attachment mechanism is operable to engage an article of clothing.

2. The hand warmer of claim 1, wherein at least one of the first and second sides include:
a breathable moisture reducing layer;
an insulating layer; and
a wind and water resisting layer.

3. The hand warmer of claim 1, wherein the attachment mechanism includes at least one of: buttonholes, magnets, a clip, snaps and fabric loops.

4. A hand warmer, comprising:
a first side;
a second side opposite from the first side, wherein the first and second side together from an enclosure adapted to receive a human hand;
a closed end;
an open end opposite the closed end, wherein the open end includes an opening sufficient to allow entry of a human hand into the enclosure;
an attachment mechanism operable to attach the hand warmer to the clothing of a user.

5. The hand warmer of claim 4, wherein the attachment mechanism is disposed on an attachment flap, the attachment flap being connected to the first side proximate to the opening.

6. The hand warmer of claim 4, wherein the attachment mechanism includes at least one buttonhole adapted to receive buttons disposed on an article of clothing.

7. The hand warmer of claim 4, wherein the attachment mechanism includes a plurality of fabric loops adapted to engage hooks disposed on an article of clothing.

8. The hand warmer of claim 4, wherein the attachment mechanism includes at least one snap designed to reversibly attach to a corresponding snap on an article of clothing.

9. The hand warmer of claim 4, wherein the attachment mechanism includes at least one clip designed to reversibly attach to an article of clothing.

10. The hand warmer of claim 4, wherein the attachment mechanism includes a magnet embedded in the attachment flap that engages a magnet embedded in the first side of the hand warmer.

11. The hand warmer of claim 4, wherein the attachment mechanism includes a strap operable to engage an arm of the user of the hand warmer.

12. The hand warmer of claim 4, further comprising:
a flap connected to the second end proximate to the opening.

13. The hand warmer of claim 4, wherein at least one of the first and second sides includes at least one of:
a breathable moisture-reducing layer;
an insulating layer; and
a wind and water resisting layer.

14. The hand warmer of claim 4, further comprising a flap portion attached to one side of the hand warmer adapted to extend to a position visible outside an article of clothing worn by a user of the hand warmer.

15. The hand warmer of claim 4, wherein the hand warmer is adapted to be positioned under a shirt, comprising a plurality of buttons, of a user, and wherein the attachment mechanism is connected to at least one of the shirt buttons.

16. The hand warmer of claim 4, wherein the hand warmer is adapted to be positioned in the back pant pocket of a user, wherein the back pant pocket includes at least one pocket button operable to close the pocket, and wherein the attachment mechanism is connected to the at least one pocket button.

17. The hand warmer of claim 4, wherein the attachment mechanism includes a strap disposed on the hand warmer adapted to cling to an arm of the user.
